(19) United States
(12) Patent Application Publication
(10) Pub. No.: US 2012/0296212 A1
(43) Pub. Date: Nov. 22, 2012

(54) ULTRASOUND DIAGNOSTIC APPARATUS, CONTROL METHOD, AND IMAGE PROCESSING APPARATUS

(75) Inventors: Kenji Hamada, Otawara-shi (JP); Cong Yao, Otawara-shi (JP)

(73) Assignees: TOSHIBA MEDICAL SYSTEMS CORPORATION, Otawara-shi (JP); KABUSHIKI KAISHA TOSHIBA, Minato-ku (JP)

(21) Appl. No.: 13/561,120

(22) Filed: Jul. 30, 2012

Related U.S. Application Data
(63) Continuation of application No. PCT/JP2011/074203, filed on Oct. 20, 2011.

(30) Foreign Application Priority Data
Oct. 20, 2010 (JP) 2010-235843

(51) Int. Cl. A61B 8/14

(52) U.S. Cl. 600/443

(57) ABSTRACT

An ultrasound diagnostic apparatus according to an embodiment includes an extracting unit, a detecting unit, and a display controlling unit. The extracting unit extracts a cervical image region that is a region including the cervical region from an ultrasonic image of a fetus obtained by transmissions and receptions of ultrasonic waves. The detecting unit detects a dorsal body surface region that is a region that is related to a dorsal body surface of the fetus from the ultrasonic image. The display controlling unit controls to display an enlarged image including an enlarged image of the cervical image region in a region other than the dorsal region in the ultrasonic image on a display device.

### CROSS-REFERENCE TO RELATED APPLICATIONS

This application is a continuation of PCT international application Ser. No. PCT/JP2011/074203 filed on Oct. 20, 2011 which designates the United States, and which claims the benefit of priority from Japanese Patent Application No. 2010-235843, filed on Oct. 20, 2010; the entire contents of which are incorporated herein by reference.

### FIELD

Embodiments described herein relate generally to an ultrasound diagnostic apparatus, a control method, and an image processing apparatus.

### BACKGROUND

Known as a method for checking for a possibility of a genetic disorder is a nuchal translucency (NT) measurement method in which the thickness of the fetal NT is measured. An NT is a swelling formed on the back of the neck of a fetus during early pregnancy, and is also referred to as a cervical edema. It is said that, when the NT is thick, the possibility of fetus abnormalities such as chromosomal abnormalities or congenital disorders including Down syndrome increases.

In the NT measurement method, a user takes an ultrasonic image of a fetus using an ultrasound diagnostic apparatus, and measures the NT thickness from the ultrasonic image thus taken. In the NT measurement method, various measurement conditions need to be satisfied. For example, in the NT measurement method, the measurement precision of 0.1 millimeter is required, and the gestational age (GA) of the fetus needs to fall within the eleven weeks to the thirteen weeks plus six days. In addition, in the NT measurement method, for example, the crown rump length (CRL) of 45 millimeters to 84 millimeters is required, and the posture of the fetus needs to satisfy certain conditions.

However, the NT thickness is sometimes not measured appropriately by the user.

### BRIEF DESCRIPTION OF THE DRAWINGS

FIG. 1 is a schematic illustrating an example of a structure of an ultrasound diagnostic apparatus according to a first embodiment.

FIG. 2 is a schematic illustrating a fetal NT.

FIG. 3 is a schematic for briefly giving the overall view of a series of processes performed by a controlling unit according to the first embodiment.

FIG. 4 is a block diagram illustrating an example of a configuration of the controlling unit included in the ultrasound diagnostic apparatus according to the first embodiment.

FIG. 5 is a schematic illustrating the dorsal body surface of a fetus.

FIG. 6 is a schematic for explaining an NT measuring unit according to the first embodiment.

FIG. 7 is a flowchart illustrating an example of a process performed by the ultrasound diagnostic apparatus according to the first embodiment.

FIG. 8 is a schematic illustrating advantageous effects achieved by the ultrasound diagnostic apparatus according to the first embodiment.

FIGS. 9 and 10 are schematics for explaining a variation according to the first embodiment.

FIGS. 11 to 14 are schematics for explaining an ultrasound diagnostic apparatus according to a second embodiment.

### DETAILED DESCRIPTION

An ultrasound diagnostic apparatus according to an embodiment includes an extracting unit, a detecting unit, and a display controlling unit. The extracting unit extracts a cervical image region that is a region including the cervical region from an ultrasonic image of a fetus obtained by transmissions and receptions of ultrasonic waves. The detecting unit detects a dorsal body surface region that is a region that is related to a dorsal body surface of the fetus from the ultrasonic image. The display controlling unit controls to display an enlarged image including an enlarged image of the cervical image region in a region other than the dorsal region in the ultrasonic image on a display device.

An example of an ultrasound diagnostic apparatus, a control method, and an image processing apparatus according to embodiments will now be explained.

### First Embodiment

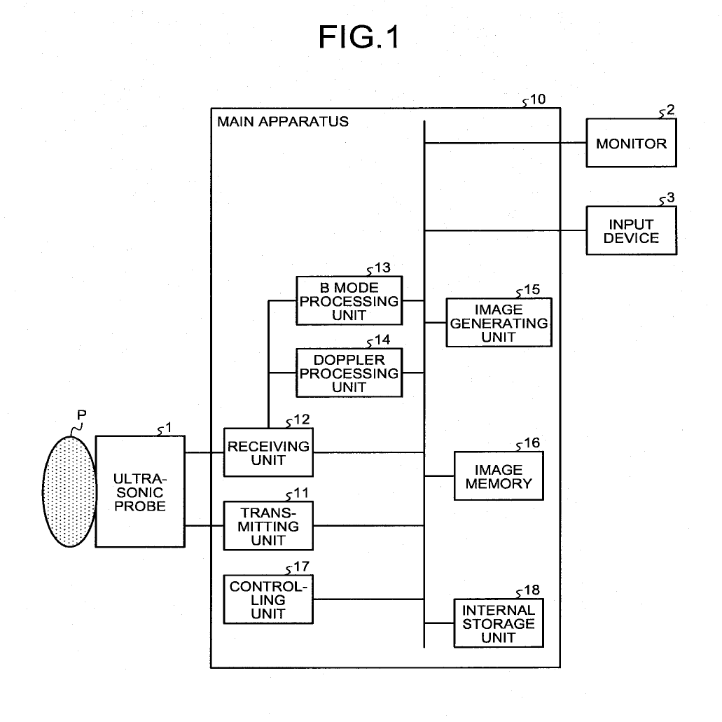

A structure of an ultrasound diagnostic apparatus according to the first embodiment will now be explained. FIG. 1 is a schematic illustrating an example of a structure of the ultrasound diagnostic apparatus according to the first embodiment. As illustrated in FIG. 1, the ultrasound diagnostic apparatus according to the first embodiment includes an ultrasonic probe 1, a monitor 2, an input device 3, and a main apparatus 10. The overall structure of the ultrasound diagnostic apparatus will be explained below to begin with, and a detailed description thereof will follow.

The overall structure of the ultrasound diagnostic apparatus will now be explained. The ultrasonic probe 1 includes a plurality of piezoelectric transducers, matching layers placed on the piezoelectric transducers, and a backing material for preventing the ultrasonic waves emitted from the piezoelectric transducers from propagating backwardly. The piezoelectric transducers included in the ultrasonic probe 1 generate ultrasonic waves based on driving signals supplied by a transmitting unit 11 included in a main apparatus 10 to be described later. The piezoelectric transducers in the ultrasonic probe 1 also receive reflection waves from a subject P, and convert the reflection waves into electrical signals. The reflection waves are also referred to as “ultrasonic wave echoes”, “echo signals”, or “reflection wave signals”.

When ultrasonic waves are transmitted from the ultrasonic probe 1 toward the subject P, the transmitted ultrasonic waves are reflected one after another on an acoustic impedance discontinuous surface of body tissues in the subject P, and received as reflection wave signals by the piezoelectric transducers included the ultrasonic probe 1. The amplitude of the reflection wave signals depends on the acoustic impedance difference in the discontinuous surface that reflects the ultrasonic waves. If an ultrasonic wave reflects on a blood flow or a surface of a cardiac wall of a beating heart, the frequency of the reflection wave signal is shifted due to the Doppler shift. At this time, the degree of frequency transition depends on the components of the velocities of the blood flow or the surface of the cardiac wall with respect to the direction in which the ultrasonic waves are transmitted.

The monitor 2 displays a graphical user interface (GUI) for allowing a user of the ultrasound diagnostic apparatus to input various settings using the input device 3, and displays an ultrasonic image generated by the main apparatus 10, for example. The monitor 2 is also referred to as “display device”. In the explanation below, the monitor 2 is explained to be a part of the ultrasound diagnostic apparatus. However, the monitor 2 is not limited thereto, and may be an external apparatus that is separate from the ultrasound diagnostic apparatus. The monitor 2 may also function as the input device 3 to be explained later.

The input device 3 includes a mouse, a keyboard, a button, a panel switch, a touch command screen, a foot switch, and a track ball. The input device 3 receives various settings from the user of the ultrasound diagnostic apparatus, and transmits the various settings thus received to the main apparatus 10. For example, the input device 3 receives an operation for specifying an approximate center of an NT image region to be explained later from the user, and transmits the operation content thus accepted to the main apparatus 10.

The main apparatus 10 generates an ultrasonic image based on the reflection wave signals received by the ultrasonic probe 1. In the example illustrated in FIG. 1, the main apparatus 10 includes a transmitting unit 11, a receiving unit 12, a B mode processing unit 13, a Doppler processing unit 14, an image generating unit 15, an image memory 16, a controlling unit 17, and an internal storage unit 18.

The transmitting unit 11 includes a trigger generator circuit, a delay circuit, and a pulser circuit. The transmitting unit 11 supplies driving signals to the ultrasonic probe 1. The transmitting unit 11 controls transmission directivity of the ultrasonic wave transmissions. More specifically, the pulser circuit repeatedly generates a rate pulse for forming transmission ultrasonic waves at a predetermined rate frequency. The delay circuit converges the ultrasonic waves generated by the ultrasonic probe 1 into a beam-like form, and adds a delay time that is required in determining the transmission directivity of each of the piezoelectric transducers to each of the rate pulses generated by the pulser circuit. The trigger generator circuit applies the driving signals to the ultrasonic probe 1 at a timing determined by the rate pulse. The driving signal is also referred to as “driving pulse”.

The receiving unit 12 includes an amplifier circuit, an analog-to-digital (A/D) converter, and an adder. The receiving unit 12 generates reflection wave data by performing various processes to the reflection wave signals received by the ultrasonic probe 1. The receiving unit 12 also controls reception directivity of the ultrasonic wave receptions.

More specifically, the amplifier circuit amplifies the reflection wave signals, and performs a gain correction process. The A/D converter A/D-converts the reflection wave signals having gain adjusted, and adds a delay time that is required in determining the directivity of the receptions. The adder generates reflection wave data by performing an addition to the reflection wave signal processed by the A/D converter. At this time, as a result of the addition performed by the adder, reflection components from the direction corresponding to the reception directivity of the reflection wave signals are emphasized.

The B mode processing unit 13 receives the reflection wave data from the receiving unit 12, and generates data for expressing the signal intensity in the brightness of the pixel (B mode data) by performing logarithmic amplification, an envelope detecting process, and the like. The data generated by the B mode processing unit 13 is also referred to as “B mode data”.

The Doppler processing unit 14 frequency-analyses velocity information in the reflection wave data received from the receiving unit 12, extracts blood flow, tissue, and contrast agent echo components affected by the Doppler shift, and generates data that is an extraction of moving object information such as an average velocity, a distribution, and power at a plurality of points. The data generated by the Doppler processing unit 14 is also referred to as “Doppler data”.

The image generating unit 15 generates an ultrasonic image from the B mode data generated by the B mode processing unit 13 and the Doppler data generated by the Doppler processing unit 14. More specifically, the image generating unit 15 converts a signal row in a scan line of an ultrasonic scan into a signal row in a scan line in a video format, which is exemplified by a television, to generate an ultrasonic image as an image to be displayed. For example, the image generating unit 15 generates a B mode image from the B mode data, and generates a Doppler image from the Doppler data. The process of converting into a signal row in a scan line in a video format performed by the image generating unit 15 is also referred to as a “scan conversion”. The B mode data and the Doppler data are also referred to as “raw data”. The image generating unit 15 may also superimpose character information including various parameters, a scale, and a body mark to the ultrasonic image thus generated to generate a superimposed image.

The image memory 16 stores therein the ultrasonic image generated by the image generating unit 15 and the superimposed image generated by performing imaging processes to the ultrasonic image. The image memory 16 may also store therein the raw data itself.

The controlling unit 17 controls the entire processes performed by the ultrasound diagnostic apparatus. More specifically, the controlling unit 17 controls the processes performed by the transmitting unit 11, the receiving unit 12, the B mode processing unit 13, the Doppler processing unit 14, and the image generating unit 15 based on various settings input by the user via the input device 3, and various control programs and various setting information read from the internal storage unit 18. As will be explained in detail later, the controlling unit 17 displays the ultrasonic image stored in the image memory 16 onto the monitor 2.

The internal storage unit 18 stores therein various data such as control programs for performing transmissions and receptions of the ultrasonic waves, imaging processes, and displaying processes, information indicating the conditions at the time when the ultrasonic image is generated by the image generating unit 15, a diagnostic protocol, and various setting information. As the information indicating the conditions at the time when the ultrasonic image is generated by the image generating unit 15, for example, the internal storage unit 18 stores therein fetal diagnosis information, and a measurement precision used when the ultrasonic image is generated by the image generating unit 15. As the measurement precision, for example, the internal storage unit 18 stores therein the number of digits or units to be displayed. The GA of the fetus, for example, corresponds to the fetal diagnosis information.

The information stored in the internal storage unit 18 and indicating the conditions at the time when the ultrasonic image is generated by the image generating unit 15 may be input by a user, or may be input by the image generating unit 15. The internal storage unit 18 may also store therein the data stored in the image memory 16. The data stored in the internal storage unit 18 may be transferred to an external peripheral device via an interface circuit not illustrated.

The overall structure of the ultrasound diagnostic apparatus according to the first embodiment is as explained above. Using such a structure, the ultrasound diagnostic apparatus according to the first embodiment generates an ultrasonic image of a fetus, and is used to allow a user to measure the thickness of a fetal NT (cervical edema).

More specifically, the ultrasound diagnostic apparatus according to the first embodiment includes an extracting unit, a detecting unit, and a display controlling unit. The extracting unit extracts a cervical image region that is a region including a cervical region from an ultrasonic image of a fetus obtained by transmissions and receptions of ultrasonic waves. The detecting unit detects a dorsal body surface region that is a region related to a dorsal body surface of the fetus from the ultrasonic image of the fetus. The display controlling unit controls to arrange an enlarged image including a part or a whole of an enlarged image of the cervical image region in a region other than the dorsal body surface region in the ultrasonic image, and displays the image onto the display device.

As is known, a fetal NT is an edema formed on the cervical region. Therefore, the ultrasound diagnostic apparatus according to the first embodiment extracts a cervical image region that is a region including the cervical region from an ultrasonic image of a fetus. In the first embodiment, the cervical image region extracted by the extracting unit is referred to as an “NT image region”. In the example explained in the first embodiment, the display controlling unit generates an enlarged image by simply enlarging the cervical image region extracted by the extracting unit. In the example in the first embodiment, an example of NT formed on the fetus is explained. However, the ultrasound diagnostic apparatus explained herein can also be used to diagnose a fetus without any formation of the NT.

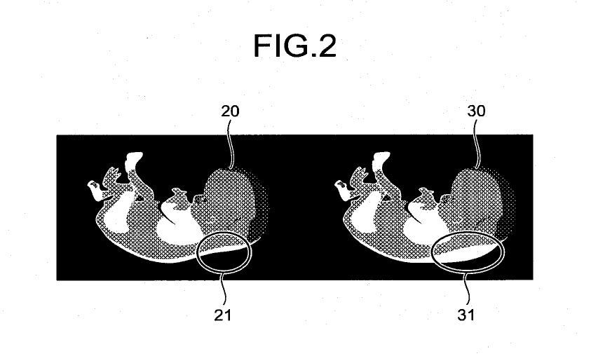

The fetal NT will be explained with reference to FIG. 2. FIG. 2 is a schematic illustrating a fetal NT. 20 and 30 in FIG. 2 represent a fetus. 21 and 31 represent a fetal NT. The fetus 20 is illustrated as an example of a normal fetus without a thick NT. The fetus 30 is illustrated as an example of a fetus with a thicker NT compared with that formed on the normal fetus. It is said that, for the fetus 30 with a thicker NT compared with the normal fetus 20, the possibility of fetus abnormalities such as chromosomal abnormalities or congenital disorders including Down syndrome increases.

At this time, for performing a NT measurement, various measurement conditions need to be satisfied. For example, because the thickness of the NT changes depending on the posture of the fetus, the posture of the fetus needs to be at a predetermined posture. However, a user might measure the thickness of the NT without satisfying the measurement conditions. As a result, the thickness of the NT thus measured may not take a valid value, or a measurement error may increase. Therefore, in the ultrasound diagnostic apparatus according to the first embodiment, the controlling unit 17 performs a series of processes explained below.

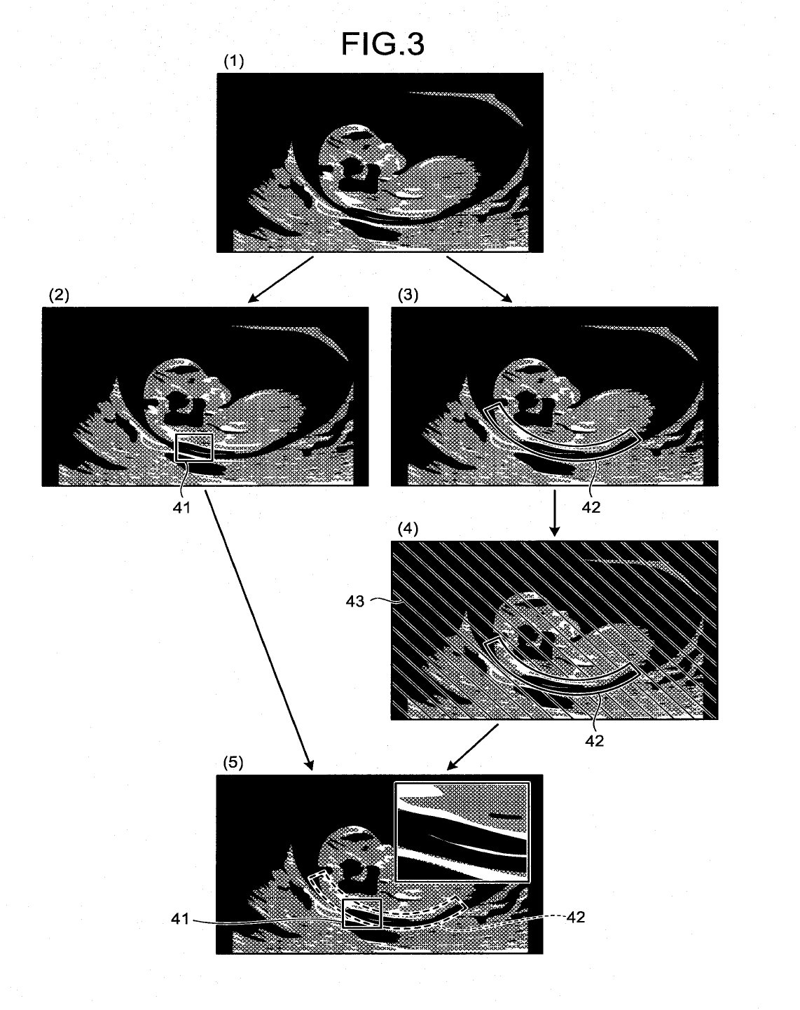

FIG. 3 is a schematic for giving an overall view of a series of processes performed by the controlling unit according to the first embodiment. A portion (1) in FIG. 3 illustrates an ultrasonic image generated by the image generating unit 15. As illustrated in a portion (2) in FIG. 3, in the controlling unit 17, an extracting unit 17a, which is to be explained later, extracts an NT image region 41 that is a region including an NT from the ultrasonic image. A detecting unit 17b detects a dorsal body surface 42 of the fetus from the ultrasonic image as illustrated in a portion (3) in FIG. 3, and detects a dorsal body surface excluded image region 43 that is a region other than the dorsal body surface 42 thus detected from the ultrasonic image as illustrated in a portion (4) in FIG. 3. In the portion (4) illustrated in FIG. 3, the hatched area corresponds to the dorsal body surface excluded image region 43. As illustrated in a portion (5) in FIG. 3, a display controlling unit 17e then displays an enlarged image of the NT image region 41 in the dorsal body surface excluded image region 43. In the portion (5) illustrated in FIG. 3, for the convenience of the explanation, the area corresponding to the dorsal body surface 42 of the fetus is surrounded by a dotted line. However, the dotted line corresponding to the dorsal body surface 42 of the fetus is not displayed on the actual screen.

In other words, by focusing on the fact that the posture of the fetus can be identified by looking at the dorsal body surface of a fetus, the extracting unit 17a displays the enlarged image of the NT image region in the dorsal body surface excluded image region, so that the dorsal body surface of the fetus is not hidden thereby. As a result, the user can visually check the enlarged image of the NT image region while looking at the posture of the fetus, and therefore, the NT measurement can be performed appropriately.

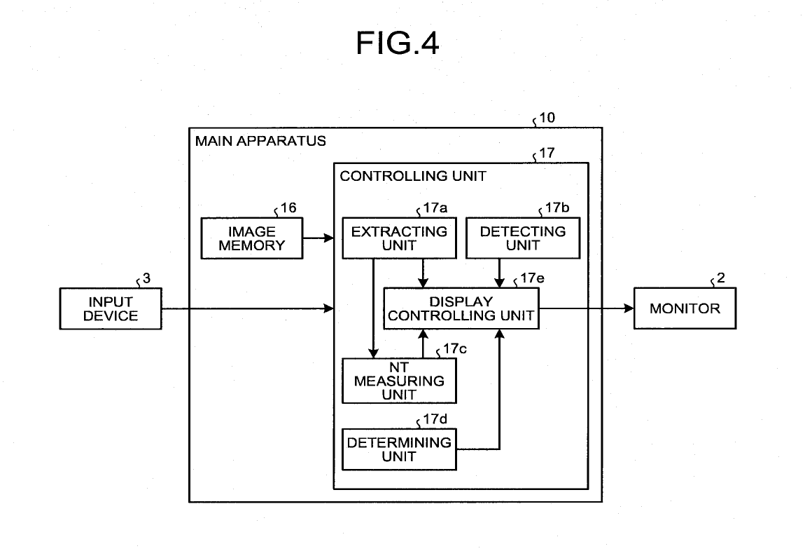

A detailed example of a configuration of the controlling unit 17 will now be explained with reference to FIG. 4. FIG. 4 is a block diagram illustrating an example of a configuration of the controlling unit included in the ultrasound diagnostic apparatus according to the first embodiment. In FIG. 4, for the convenience of the explanation, the monitor 2, the input device 3, the main apparatus 10, and the image memory 16 are illustrated as well. As illustrated in FIG. 4, the controlling unit 17 includes an extracting unit 17a, a detecting unit 17b, an NT measuring unit 17c, a determining unit 17d, and a display controlling unit 17e.

The extracting unit 17a extracts the NT image region from the ultrasonic image generated by the image generating unit 15. For example, when an approximate center of the NT image region is specified by the user, the extracting unit 17a extracts a part of the image that is within a predetermined range from the area thus specified as an NT image region. Because it is known that the region where the NT is formed is on the back of the neck of a fetus, the extracting unit 17a may perform a known image recognizing process to detect a part corresponding to the back of the neck of the fetus from the ultrasonic image, and extract the part thus detected as the NT image region.

The extracting unit 17a may extract an image region in any shape as the NT image region. For example, the extracting unit 17a may extract a circular, an elliptical, a rectangular, or a square image region. Furthermore, the extracting unit 17a may receive a setting of the size of the image region that is to be extracted as the NT image region from the user after the approximate center of the NT image region is specified by the user. For example, if the NT image region is circular, the extracting unit 17a may receive a radius from the user.

The detecting unit 17b detects the dorsal body surface of the fetus from the ultrasonic image, and detects the dorsal body surface excluded image region that is a region not including the dorsal body surface thus detected from the ultrasonic image. For example, the detecting unit 17b performs a known image recognizing process to detect the dorsal body surface of the fetus, and detects the remaining region of the ultrasonic image not including the region thus detected as the dorsal body surface excluded image region. For detecting the dorsal body surface excluded image region, the detecting unit 17b may receive a specification of an image area including the dorsal body surface excluded image region from the user, and detects the image region thus specified as the dorsal body surface excluded image region.

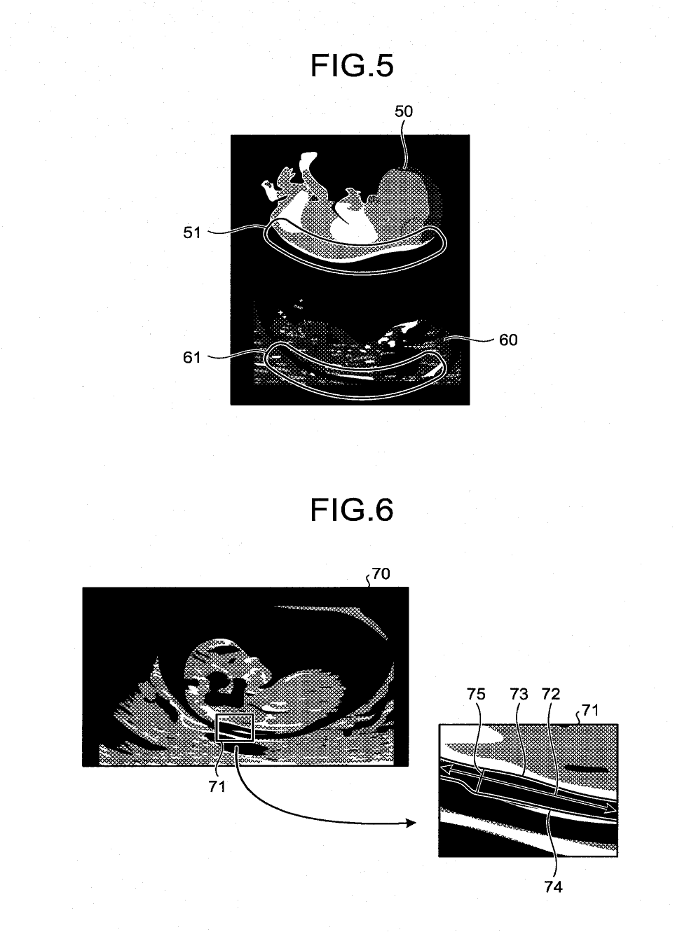

The dorsal body surface of the fetus will be explained with reference to FIG. 5. FIG. 5 is a schematic illustrating the dorsal body surface of the fetus. 50 in FIG. 5 represents an example of a fetus, and 60 represents an example of an ultrasonic image of the fetus 50, and 51 and 61 represent the dorsal body surface of the fetus. As illustrated in FIG. 5, the dorsal body surface of the fetus is a body surface on the back of the fetus. In other words, the dorsal body surface of the fetus is a portion of the contour on the back of the fetus included in the ultrasonic image.

The NT measuring unit 17c performs an NT measurement by detecting a longitudinal direction of the NT in the NT image region extracted by the extracting unit 17a, detecting boundaries along directions perpendicular to the longitudinal direction thus detected, and measuring the distance between the boundaries thus detected.

As a method for detecting the longitudinal direction of the NT, for example, the NT measuring unit 17c searches for a low luminance part from the central point of the NT image region in the radial directions, and detects the direction extending in the longest path as the longitudinal direction. In other words, the NT measuring unit 17c detects a low luminance part along every given line including the central point, and calculates the distance between the low luminance parts thus detected. The NT measuring unit 17c then detects the line whose calculated distance is the longest as the longitudinal direction.

The NT measuring unit 17c also detects the boundaries between the NT and the regions other than NT by calculating the difference in adjacent pixel values, for example. At this time, the NT measuring unit 17c performs the NT measurement for measuring the thickness of the NT by measuring the length of a line segment perpendicularly crossing the longitudinal direction thus detected and laid between the boundaries between the NT and the region other than NT.

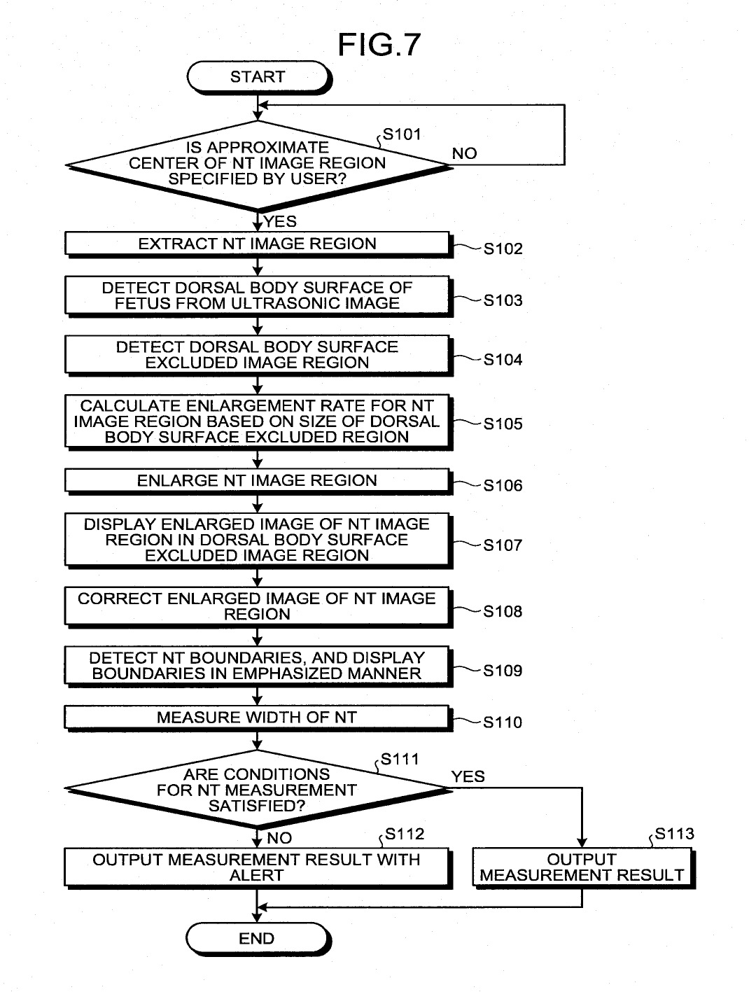

FIG. 6 is a schematic for explaining the NT measuring unit according to the first embodiment. 70 in FIG. 6 represents an ultrasonic image. 71 in FIG. 6 represents an enlarged image of the NT image region. 72 in FIG. 6 represents the longitudinal direction detected by the NT measuring unit 17c. 73 and 74 in FIG. 6 represent the boundaries detected by the NT measuring unit 17c. In the example illustrated in FIG. 6, the NT measuring unit 17c detects boundaries along each of lines perpendicularly crossing the longitudinal direction at every point on the line along the longitudinal direction.

75 in FIG. 6 is a line segment perpendicularly crossing the longitudinal direction and having each end on the boundary 73 and the boundary 74. As a result, the length of the line segment 75 represents the thickness of the NT. As illustrated in FIG. 6, the NT measuring unit 17c calculates the thickness of the NT by detecting the longitudinal direction 72 in the NT image region, detecting intersecting points between a direction perpendicularly crossing the longitudinal direction 72 and the boundary 73 and the boundary 74, and calculating the distance (maximum distance) between the intersecting points thus detected.

The determining unit 17d determines if the conditions at the time when the ultrasonic image is generated by the image generating unit 15 satisfy conditions required to be satisfied for performing the NT measurement. For example, the determining unit 17d obtains the conditions at the time when the ultrasonic image is generated by the image generating unit 15 by reading such conditions from the internal storage unit 18, and analyzing the ultrasonic image. To explain using a more specific example, the determining unit 17d reads the GA and the measurement precision from the internal storage unit 18, and obtains the CRL of the fetus by analyzing the ultrasonic image. The determining unit 17d then determines if the measurement precision of the thickness of the NT measured by the NT measuring unit 17c is 0.1 millimeter, determines if the GA of the fetus falls within the eleven weeks to the thirteen weeks plus six days, and determines if the CRL of the fetus is 45 millimeters to 84 millimeters.

The display controlling unit 17e controls to display the ultrasonic image as well as the enlarged image that is an enlargement of the NT image region extracted by the extracting unit 17a on the monitor 2. More specifically, the display controlling unit 17e displays the enlarged image of the NT image region in the dorsal body surface excluded image region detected by the detecting unit 17b (see (5) in FIG. 3).

The thickness of the NT changes depending on the posture of the fetus, and for performing an NT measurement, it is important to understand the posture of the fetus. At this time, based on the fact that the posture of the fetus can be identified by looking at the dorsal body surface of the fetus, the display controlling unit 17e displays the enlarged image of the NT image region in the dorsal body surface excluded image region so that the dorsal body surface of the fetus is not hidden thereby.

The display controlling unit 17e calculates an enlargement rate at which the NT image region fits in the dorsal body surface excluded image region detected by the detecting unit 17b, and enlarges the NT image region at the enlargement rate thus calculated. For example, the display controlling unit 17e calculates the maximum enlargement rate at which the NT image region fits in the dorsal body surface excluded image region, and enlarges the NT image region using the maximum enlargement rate thus calculated. When an upper limit is specified by the user as the enlargement rate in advance, and if the maximum enlargement rate thus calculated exceeds such an upper limit, for example, the display controlling unit 17e enlarges the NT image region using the enlargement rate at the upper limit. The display controlling unit 17e may also be specified with different maximum enlargement rates depending on the size in which the image before being enlarged is displayed. The upper limit may also be determined based on the performance of the ultrasonic probe 1 or the performance of the monitor 2. As the upper limit, an enlargement rate at which “one centimeter” in the ultrasonic image is represented as a half of the monitor 2, for example, may be used.

The position where the enlarged image is arranged may be changed by the user. An example of determining the position for the enlarged image will now be explained. For example, the display screen may be divided into equal four sections, and the enlarged image may be displayed in the section located diagonally to the position where the NT image region is assigned by the user. An example in which the display screen is divided into four sections consisting of two columns and two rows will now be explained. In this example, if the area including the NT image region is assigned to a section located at the second row and the second column, the display controlling unit 17e displays the enlarged image at the section located at the first row and the first column. In other words, if the NT image region is assigned to the lower left section by the user, the display controlling unit 17e displays the enlarged image at the upper right section that is located diagonally to the NT image region.

The display controlling unit 17e also controls to display a measurement result received from the NT measuring unit 17c together with the enlarged image of the NT image region and the ultrasonic image. At this time, if the determining unit 17d determines that the conditions are not satisfied, the display controlling unit 17e outputs an alert to the user. To explain using a more specific example, if the determining unit 17d determines that the conditions are not satisfied, the display controlling unit 17e displays the measurement result with a predetermined mark appended thereto, or displays the measurement result by changing the color of the screen. The predetermined mark corresponds to a given symbol or a given mark, such as a star, an asterisk, and an inequality sign. The display controlling unit 17e is also referred to as an “alert output unit”.

For enlarging the NT image region, the display controlling unit 17e may adjust the luminance, or may perform a blur reducing process or a boundary emphasizing process. For example, the display controlling unit 17e adjusts at least one of the enlarged image of the NT image region and the ultrasonic image so that the average luminance in the enlarged image of the NT image region and the average luminance in the ultrasonic image are within a predetermined range. To explain using a more specific example, the display controlling unit 17e adjusts the luminance of the enlarged image of the NT image region or the luminance in the ultrasonic image or adjusts the luminance of the enlarged image of the NT image region and the luminance of the ultrasonic image, to make the average luminance equivalent.

As another example of the luminance adjustment, the display controlling unit 17e may make an adjustment so that the histogram of the enlarged image in the NT image region is not changed by a large degree. The display controlling unit 17e also detects the boundaries of the NT, displays the boundary parts thus detected in an emphasized function in the enlarged image of the NT image region, as an example. To explain with reference to FIG. 6, the display controlling unit 17e displays the boundary 73 and the boundary 74 on the enlarged image of the NT image region.

Process According to First Embodiment

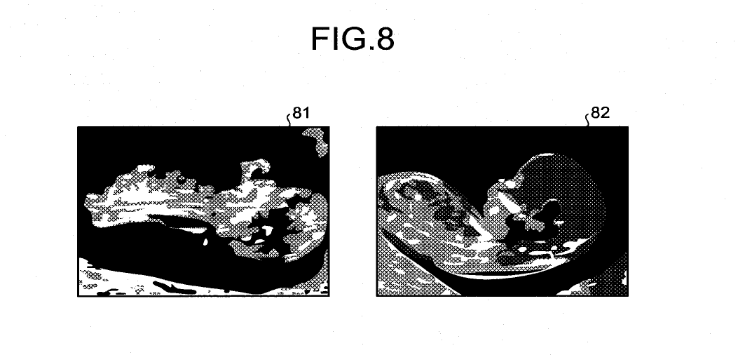

FIG. 7 is a flowchart illustrating an example of a process performed by the ultrasound diagnostic apparatus according to the first embodiment.

As illustrated in FIG. 7, when the user specifies the approximate center of the NT image region (YES at Step S101), the extracting unit 17a extracts the NT image region (Step S102). More specifically, the extracting unit 17a extracts a part of the image that is within a predetermined range from the specified area as the NT image region.

The detecting unit 17b then detects the dorsal body surface of the fetus from the ultrasonic image (Step S103), and detects the dorsal body surface excluded image region that is the region not including the dorsal body surface thus detected from the ultrasonic image (Step S104).

The display controlling unit 17e then calculates an enlargement rate for the NT image region based on the size of the dorsal body surface excluded image region (Step S105). For example, the display controlling unit 17e calculates the maximum enlargement rate at which the NT image region fits in the dorsal body surface excluded image region. The display controlling unit 17e then enlarges the NT image region at the enlargement rate thus calculated (Step S106).

The display controlling unit 17e then displays the enlarged image of the NT image region in the dorsal body surface excluded image region (Step S107). In other words, the display controlling unit 17e displays the enlarged image that is an enlargement of the NT image region extracted by the extracting unit 17a as well as the dorsal body surface of the fetus that is imaged in the ultrasonic image onto the monitor 2.

The display controlling unit 17e then corrects the enlarged image of the NT image region (Step S108). For example, the display controlling unit 17e adjusts the luminance so as to make the average luminance in the enlarged image of the NT image region and the average luminance in the ultrasonic image equivalent. The display controlling unit 17e then detects the boundaries of the NT, and displays the NT in an emphasized function (Step S109). To explain with reference to FIG. 6, for example, the display controlling unit 17e displays the boundary 73 and the boundary 74 on the enlarged image of the NT image region.

The NT measuring unit 17c measures the width of the NT (Step S110). To explain with reference to FIG. 6, the NT measuring unit 17c calculates the distance between the boundary 73 and the boundary 74 to calculate the thickness 75 of the NT.

The determining unit 17d then determines if the conditions of the NT measurement are satisfied (Step S111). More specifically, the determining unit 17d determines if the conditions of the fetus at the time when the ultrasonic image is generated by the image generating unit 15 satisfy the conditions required to be satisfied for performing the NT measurement. At this time, if the determining unit 17d determines that the conditions are not satisfied (NO at Step S111), the display controlling unit 17e outputs the measurement result with an alert (Step S112). For example, the display controlling unit 17e appends a given mark such as a star, an asterisk, and an inequality sign to the measurement result, and display the result. On the contrary, if the determining unit 17d determines that the conditions are satisfied (YES at Step S111), the display controlling unit 17e displays the measurement result as it is (Step S113).

In the example of the process illustrated in FIG. 7, the ultrasound diagnostic apparatus performs the correction to the enlarged image and performs the display emphasizing process after the enlarged image of the NT image region is displayed. However, examples are not limited thereto. For example, the ultrasound diagnostic apparatus may correct the enlarged image, perform the display emphasizing process, and then display the enlarged image applied with the correction and the display emphasizing process. Furthermore, in the example of the process illustrated in FIG. 7, the ultrasound diagnostic apparatus measures the thickness of the NT after the enlarged image of the NT image region is displayed. However, examples are not limited thereto. For example, the ultrasound diagnostic apparatus may measure the thickness of the NT before displaying the enlarged image of the NT image region.

Effects Achieved by First Embodiment

As described above, according to the first embodiment, the image generating unit 15 generates an ultrasonic image of a fetus based on the reflection waves of ultrasonic waves transmitted from the ultrasonic probe to the fetus. Furthermore, the controlling unit 17 controls to extract an NT image region from the ultrasonic image thus generated, and to display an enlarged image that is an enlarged image of the NT image region thus extracted as well as the ultrasonic image on the display device. As a result, the enlarged image of the NT image region can be displayed, and an NT measurement can be performed appropriately. More specifically, a user can measure the thickness of the NT while looking at the enlarged image of the NT. Therefore, the accuracy of the NT measurement can be improved.

Furthermore, according to the first embodiment, the controlling unit 17 detects the dorsal body surface of the fetus from the ultrasonic image, and detects the dorsal body surface excluded image region from the ultrasonic image. Furthermore, the controlling unit 17 displays the enlarged image of the NT image region in the dorsal body surface excluded image region thus detected. As a result, the NT measurement can be performed while ensuring the user to check if the posture of the fetus is a posture suitable for the NT measurement. Therefore, the NT measurement can be performed appropriately.

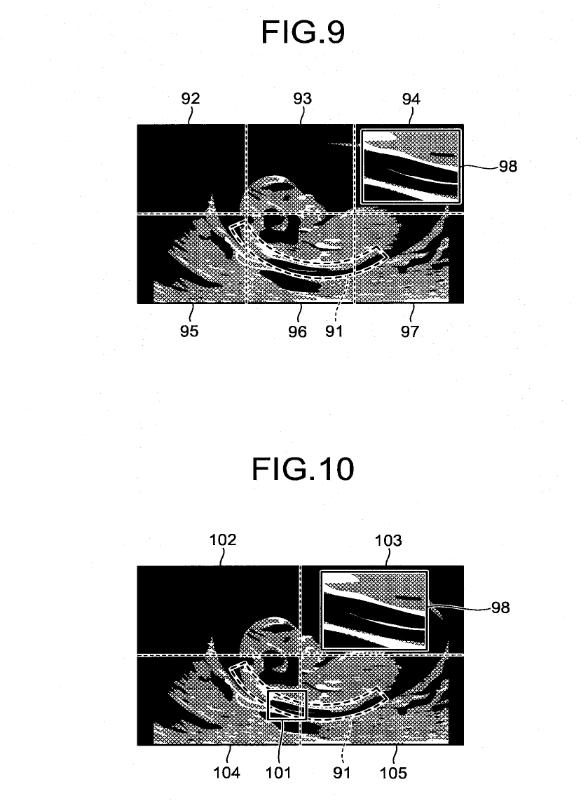

The posture of the fetus and the thickness of the NT in the first embodiment will now be explained further with reference to FIG. 8. FIG. 8 is a schematic illustrating the advantageous effects achieved by the ultrasound diagnostic apparatus according to the first embodiment. “81” and “82” in FIG. 8 are examples of the ultrasonic image of a fetus. In the fetus “81” illustrated in FIG. 8, the cervical region of the fetus is retroflex, and, in the fetus “82” in FIG. 8, the cervical region of the fetus is anteflexed. For measuring the thickness of the NT, it is required to measure the thickness while the fetus is at a neutral position. It is known that, when the cervical region of the fetus is retroflex as illustrated as “81” in FIG. 8, the thickness of the NT increases by “0.6 millimeters”, and when the cervical region of the fetus is anteflexed as illustrated as “82” in FIG. 8, the thickness of the NT decreases by “0.4 millimeters”.

As explained above with reference to FIG. 8, the thickness of the NT changes depending on the posture of the fetus. Considering the fact that, as to the posture of the fetus, it is possible to determine if the fetus is retroflex, anteflexed, or at a neutral position by looking at the dorsal body surface of the fetus, for example, the ultrasound diagnostic apparatus displays the enlarged image of the NT image region in the dorsal body surface excluded image region. As a result, the user can check the enlarged image of the NT image region and the dorsal body surface of the fetus at the same time, and therefore, the user can measure the thickness of the NT while checking the posture of the fetus.

Furthermore, according to the first embodiment, the controlling unit 17 calculates an enlargement rate at which the NT image region fits in the dorsal body surface excluded image region thus detected, and enlarges the NT image region at the enlargement rate thus calculated. As a result, the NT image region can be enlarged to an extent where the dorsal body surface of the fetus is not hidden thereby, and the thickness of the NT can be measured while allowing the posture of the fetus to be checked.

Furthermore, according to the first embodiment, when the user specifies the approximate center of the NT image region, the controlling unit 17 extracts the image part within a predetermined range from the area thus specified as an NT image region. As a result, the controlling unit 17 can extract the NT image region reliably, and the user can visually check the enlarged image of the NT image region reliably.

Furthermore, according to the first embodiment, the controlling unit 17 adjusts the luminance so as to make the average luminance in the enlarged image of the NT image region and the average luminance in the ultrasonic image equivalent. As a result, even if the ultrasonic image and the enlarged image are displayed together, these images can be displayed while allowing the user to look at both of these images comfortably.

Furthermore, according to the first embodiment, the controlling unit 17 controls to detect the boundaries of the NT, to display the boundary parts thus detected in an emphasized function in the enlarged image of the NT image region. As a result, the user can check the boundaries of the NT reliably, and measure the thickness of the NT easily.

Furthermore, according to the first embodiment, the controlling unit 17 detects the longitudinal direction of the NT in the NT image region, performs a boundary detection in the directions perpendicular to the longitudinal direction thus detected, and measures the distance between the boundaries thus detected. As a result, processing burdens of the user can be reduced for measuring the thickness of the NT.

Furthermore, according to the first embodiment, the controlling unit 17 controls to display the measurement result in combination with the enlarged image of the NT image region and the ultrasonic image. As a result, the measurement result can be examined while allowing the enlarged image of the NT image region and the ultrasonic image to be checked.

Furthermore, according to the first embodiment, the controlling unit 17 determines if the conditions of the fetus at the time when the ultrasonic image is generated satisfies the conditions that are required to be satisfied for performing an NT measurement, and, if the controlling unit 17 determines that the conditions are not satisfied, the controlling unit 17 outputs an alert to the user. As a result, even if the thickness of the NT is measured although the GA or the CRL of the fetus is not within the applicable range for the NT measurement, the user can be prevented from overlooking such conditions not being within the applicable range.

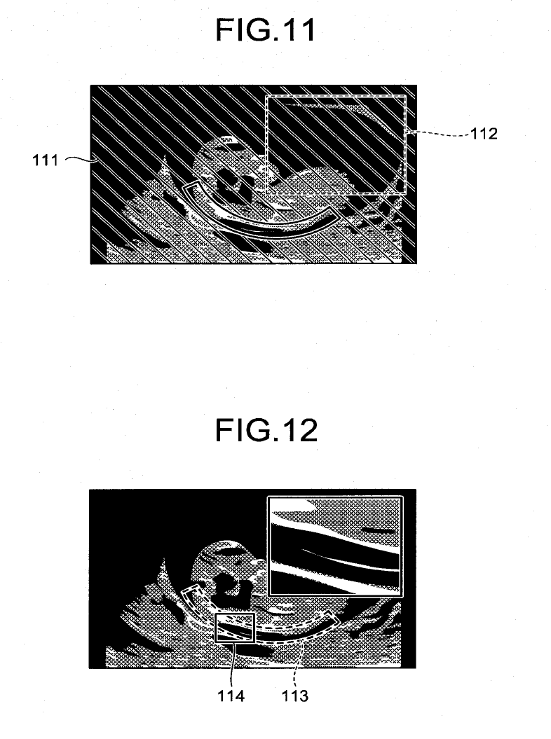

As a variation of the first embodiment, for example, the detecting unit 17b may detect the dorsal body surface region that is the region related to the dorsal body surface of the fetus from the ultrasonic image, and the display controlling unit 17e may divide the area of the ultrasonic image into a plurality of sections, and arrange the enlarged image in a section not including the dorsal body surface region among the sections thus divided. FIGS. 9 and 10 are schematics for explaining the variation according to the first embodiment.

For example, as illustrated in FIG. 9, the detecting unit 17b detects a dorsal body surface region 91 related to the dorsal body surface of the fetus from the ultrasonic image. At this time, the method by which the detecting unit 17b detects the dorsal body surface region 91 is the same as the method for detecting the body surface in the embodiment explained above. The display controlling unit 17e then divides the area of the ultrasonic image into six sections 92 to 97, for example, as illustrated in FIG. 9. At this time, in the example illustrated in FIG. 9, a part of the dorsal body surface region 91 is included in the section 95, 96, and 97. Therefore, the display controlling unit 17e controls to arrange an enlarged image 98 in one of the remaining sections 92, 93, and 94. At this time, for example, the display controlling unit 17e receives an operation of selecting one of the sections not including the dorsal body surface region from the user, and controls to display the enlarged image in the section selected by the operation.

The display controlling unit 17e may also control to divide the area of the ultrasonic image into at least four sections, and arrange the enlarged image in a section not including the dorsal region and located diagonally to the cervical image region among at least the four sections thus divided, for example. In such an example, as illustrated in FIG. 10, the display controlling unit 17e divides the area of the ultrasonic image into four sections 102 to 105, for example. At this time, the sections 104 and 105 include a part of the dorsal body surface region 91. Among the remaining sections 102 and 103, the section 103 is located diagonally to a cervical image region 101. Therefore, the display controlling unit 17e controls to arrange the enlarged image 98 in the section 103.

As another variation, the detecting unit 17b may detect a ventral region that is a ventral region of the fetus from the ultrasonic image, and the display controlling unit 17e may control to arrange the enlarged image in the ventral region in the ultrasonic image, for example. In such an example, the detecting unit 17b is controlled to detect the face of the fetus by performing a known image recognizing process, and to detect the ventral region from the position of the face of the fetus and the position of the dorsal body surface thus detected, for example. The display controlling unit 17e then controls to display the enlarged image in the ventral region detected by the detecting unit 17b.

### Second Embodiment

A second embodiment will now be explained. In the example explained in the first embodiment, the display controlling unit 17e generates an enlarged image by simply enlarging the cervical image region extracted by the extracting unit 17a. On the contrary, in the example explained in the second embodiment, the display controlling unit 17e generates an enlarged image including part of or all of the enlarged cervical image region. In other words, in the second embodiment, the display controlling unit 17e generates an image in which not only a part of the cervical image region or the cervical image region is enlarged but also regions surrounding the cervical image region is enlarged as an enlarged image. In other words, in the second embodiment, the size of the area in which the enlarged image is to be displayed and the rate at which the cervical image region is enlarged are determined separately.

The structure of the ultrasound diagnostic apparatus according to the second embodiment is basically the same as that illustrated in FIGS. 1 and 4, except that the display controlling unit 17e determines the size of the area in which the enlarged image is to be displayed and the rate at which the cervical image region is enlarged separately. Functions of the display controlling unit 17e according to the second embodiment will be explained below. The display controlling unit 17e according to the second embodiment determines the position where the enlarged image is displayed using the same method as that according to the first embodiment.

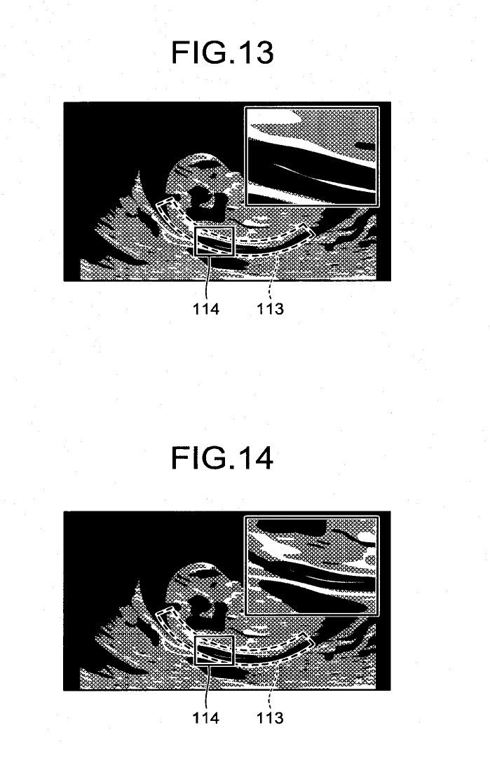

FIGS. 11 to 14 are schematics for explaining the ultrasound diagnostic apparatus according to the second embodiment. In the second embodiment, the display controlling unit 17e determines, to begin with, the size of the area in which the enlarged image is to be displayed. More specifically, the display controlling unit 17e determines the size of the area in which the enlarged image is to be displayed in a manner corresponding to the size of the dorsal body surface excluded image region that a region not including the dorsal body surface region in the ultrasonic image of the fetus.

For example, as illustrated in FIG. 11, the display controlling unit 17e determines the maximum size of the area (the size of a rectangular 112 illustrated with a dotted line in FIG. 11) in which the enlarged image can be displayed in a manner fitting in a dorsal body surface excluded image region 111 (the hatched area illustrated in FIG. 11), which is the region not including the dorsal body surface. Alternatively, the display controlling unit 17e may also determine the size obtained by multiplying a given rate to the size of the dorsal body surface excluded image region, e.g., a half or one-third of the dorsal body surface excluded image region, to be the size of the area in which the enlarged image is to be displayed.

The display controlling unit 17e then determines the rate at which the cervical image region is enlarged. More specifically, the display controlling unit 17e calculates the rate at which the cervical image region is enlarged based on the size of the dorsal body surface excluded image region and the size of the cervical image region, and enlarges the cervical image region using the enlargement rate thus calculated.

For example, the display controlling unit 17e calculates the maximum enlargement rate at which the cervical image region fits in the dorsal body surface excluded image region, and enlarges the cervical image region using the enlargement rate thus calculated. By allowing the display controlling unit 17e to determine the size in which the enlarged image is displayed as the maximum size in which the enlarged image fits in the dorsal body surface excluded image region as illustrated in FIG. 11, an entire cervical image region 114 can be displayed at the largest size without hiding a dorsal body surface region 113, in the same manner as in the first embodiment, as illustrated in FIG. 12.

If the rate at which the cervical image region 114 is enlarged is high with respect to the size of the area thus determined in which the enlarged image is to be displayed as illustrated in FIG. 13, an enlarged image of only a part of the cervical image region 114 is displayed as the enlarged image. At this time, because only a part of the cervical image region 114 is enlarged and displayed and the region of interest in the cervical image region 114 is not displayed, the display controlling unit 17e pans the enlarged image based on an operation performed by the user to display the region where the user would like to observe. “Panning” mentioned herein means, when only a part of an image to be displayed is being displayed, moving the area being displayed with respect to the image. If the rate at which the cervical image region 114 is enlarged is low with respect to the determined size in which the enlarged image is displayed as illustrated in FIG. 14, an enlarged image including the cervical image region 114 as well as regions surrounding the cervical image region 114 will be displayed. At this time, the display controlling unit 17e may change the size of the cervical image region 114 to the size of the region that is being displayed as the enlarged image, or the region being displayed as the enlarged image may be displayed as a separate region that is different from the cervical image region 114.

The display controlling unit 17e may receive the enlargement rate from the user via the input device 3 and store the enlargement rate in the internal storage unit 18, for example, and may enlarge the cervical image region using such an enlargement rate. Furthermore, the display controlling unit 17e may also receive a change instruction in the enlargement rate from the user via the input device 3, and change the enlargement rate stored in the internal storage unit 18, for example, based on the change instruction thus received.

Furthermore, for example, if the calculated enlargement rate exceeds a predetermined upper limit, the display controlling unit 17e may use such an upper limit in enlarging the image of the cervix. At this time, as the upper limit, for example, the display controlling unit 17 uses an enlargement rate at which the scale of the ultrasonic image corresponds to a predetermined scale determined by the size of the display area of the display device. For example, the display controlling unit 17e uses an enlargement rate at which “one centimeter” in the ultrasonic image corresponds to a half of the display area in the monitor 2 as an upper limit. Alternatively, the display controlling unit 17e may use the maximum enlargement rate at which the cervical image region fits in the dorsal body surface excluded image region as the upper limit of the enlargement rate.

### Third Embodiment

Some embodiments are as explained above. However, other embodiments may be implemented than those described above. Therefore, some of the other embodiments will be explained below.

### Image Correction

For example, the above embodiments are explained using an example in which the display controlling unit 17e adjusts the luminance and the like in the enlarged image of the NT image region. However, embodiments are not limited thereto. For example, the display controlling unit 17e may display the enlarged image of the NT image region as it is without adjusting the luminance and the like.

Displaying in Emphasized Function

Furthermore, for example, the above embodiments are explained using an example in which the display controlling unit 17e detects the boundaries of the NT, and displays the boundaries thus detected in an emphasized function. However, embodiments are not limited thereto. For example, the display controlling unit 17e may not display in an emphasized function.

NT Measurement

Furthermore, for example, the above embodiments are explained using an example in which the NT measuring unit 17c detects the boundaries of the NT, and measures the thickness of the NT automatically. However, embodiments are not limited thereto. For example, the user may measure the thickness of the NT manually, or the user may correct the measurement result of the NT measuring unit 17c. To explain using a specific example, when the user specifies two points on the boundaries of the NT, the NT measuring unit 17c may measure the distance between the two points thus specified, and output the measurement result to the user. Furthermore, in this example, the display controlling unit 17e controls, for example, to display the result of the NT measurement performed based on the enlarged image by the user together with the enlarged image and the ultrasonic image. Alternatively, while the display controlling unit 17e displays the measurement result of the NT measurement, the display controlling unit 17e may not display one of or both of the enlarged image and the ultrasonic image. Furthermore, for example, a correction instruction for correcting the positions of the boundaries detected by the NT measuring unit 17c may be received from the user.

System Configuration

Furthermore, a part or a whole of each of the processes explained to be performed automatically in the embodiments may be performed manually, or a part or a whole of the processes explained to be performed manually can be performed automatically according to known methods. For example, the NT image region may be extracted automatically from the ultrasonic image by performing a known image recognizing process. Processing or controlling procedures, specific names, and information including various types of data and parameters mentioned herein or in the drawings (FIGS. 1 to 8) may be modified in any way except where specified otherwise.

Furthermore, each of the element illustrated in the drawings are schematic depiction of their functionality, and does necessary not have to be configured physically in the manner illustrated in the drawings. In other words, specific configurations in which the apparatuses are distributed or integrated are not limited to those illustrated in the drawings, and a whole or a part of the apparatuses may be distributed or integrated functionally or physically in any units depending on various loads or utilization. For example, to explain using the example in FIG. 1 and FIG. 4, the extracting unit 17a to the display controlling unit 17e in the controlling unit 17 may be configured as an external apparatus, and may be connected to the ultrasound diagnostic apparatus over a network.

In other words, the above embodiments are explained using the example in which the ultrasound diagnostic apparatus performs the image processing to the ultrasonic image. However, embodiments are not limited thereto. For example, in FIG. 4, an image processing apparatus including the controlling unit 17 may receive an ultrasonic image from the ultrasound diagnostic apparatus, and executes the series of processes. For example, the image processing apparatus may receive the ultrasonic image from a database in a picture archiving and communication system (PACS) that are systems for managing various medical image data, and perform the series of processes thereto, or receive the ultrasonic image from a database in an electronic medical record system for managing electronic medical records having an attachment of medical images, and perform the series of processes thereto.

The image processing apparatus may be realized using any known information processing system such as a personal computer, a work station, or a personal digital assistant (PDA). For example, the image processing apparatus may be realized by installing the controlling unit 17, the monitor 2, and the image memory 16 illustrated in FIG. 4 in an information processing system such as a PDA.

Others

The control programs executed on the ultrasound diagnostic apparatus according to the embodiments may be distributed over a network such as the Internet. Furthermore, the ultrasound diagnostic apparatus program may be provided in a manner recorded in a computer-readable recording medium, such as a hard disk, a flexible disk (FD) a compact disk read-only memory (CD-ROM), a magneto-optical disk (MO), and a digital versatile disk (DVD), and be executed by causing a computer to read the program from the recording medium.

Effects Achieved by Embodiments

As explained above, according to the embodiments, an NT measurement can be performed appropriately.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnostic apparatus comprising:
an extracting unit configured to extract a cervical image region that is a region including a cervical region from an ultrasonic image of a fetus obtained by transmission and reception of ultrasonic waves;
a detecting unit configured to detect a dorsal body surface region that is a region related to a dorsal body surface of the fetus from the ultrasonic image; and
a display controlling unit configured to control to arrange an enlarged image including an enlarged image of the cervical image region in a region other than the dorsal body surface region included in the ultrasonic image, and to display the enlarged image onto a display device.

2. The ultrasound diagnostic apparatus according to claim 1, wherein the display controlling unit controls to divide an area of the ultrasonic image into a plurality of sections, and to arrange the enlarged image in a section not including the dorsal body surface region among such sections.

3. The ultrasound diagnostic apparatus according to claim 1, wherein the display controlling unit controls to divide an area of the ultrasonic image into at least four sections, and to arrange the enlarged image in a section not including the dorsal body surface region and positioned diagonally to the cervical image region among the at least four section.

4. The ultrasound diagnostic apparatus according to claim 1, wherein
the detecting unit further detects a ventral region that is a ventral region of the fetus from the ultrasonic image, and
the display controlling unit controls to arrange the enlarged image in the ventral region included in the ultrasonic image.

5. The ultrasound diagnostic apparatus according to claim 1, wherein the display controlling unit determines a size of an area in which the enlarged image is displayed depending on a size of a dorsal body surface excluded image region that is a region not including the dorsal body surface region in the ultrasonic image.

6. The ultrasound diagnostic apparatus according to claim 5, wherein the display controlling unit determines a maximum size in which the enlarged image is allowed to fit in the dorsal body surface excluded image region as the size of the area in which the enlarged image is to be displayed.

7. The ultrasound diagnostic apparatus according to claim 6, wherein
the display controlling unit calculates a maximum enlargement rate at which the cervical image region fits in the dorsal body surface excluded image region, and enlarges the cervical image region using the enlargement rate thus calculated.

8. The ultrasound diagnostic apparatus according to claim 1, wherein
the display controlling unit calculates an enlargement rate for the cervical image region based on a size of a dorsal body surface excluded image region that is a region not including the dorsal body surface region in the ultrasonic image and a size of the cervical image region, and enlarges the cervical image region using the enlargement rate thus calculated.

9. The ultrasound diagnostic apparatus according to claim 8, wherein the display controlling unit calculates a maximum enlargement rate at which the cervical image region is allowed to fit in the dorsal body surface excluded image region, and enlarges the cervical image region using the enlargement rate thus calculated.

10. The ultrasound diagnostic apparatus according to claim 8, wherein the display controlling unit enlarges, when the enlargement rate thus calculated exceeds a predetermined upper limit, the cervical image region using the upper limit.

11. The ultrasound diagnostic apparatus according to claim 10, wherein the display controlling unit uses, as the upper limit, an enlargement rate at which a scale in the ultrasonic image is displayed in a given scale that is determined by a size of a display area on the display device.

12. The ultrasound diagnostic apparatus according to claim 10, wherein the display controlling unit uses a maximum enlargement rate at which the cervical image region is allowed to fit in the dorsal body surface excluded image region as the upper limit.

13. The ultrasound diagnostic apparatus according to claim 1, wherein the extracting unit extracts an image part that is within a predetermined area from a center specified by a user in the ultrasonic image as the cervical image region.

14. The ultrasound diagnostic apparatus according to claim 1, wherein the display controlling unit adjusts at least one of the enlarged image and the ultrasonic image so that average luminance of the enlarged image and average luminance of the ultrasonic image are within a predetermined range.

15. The ultrasound diagnostic apparatus according to claim 1, wherein the display controlling unit detects boundaries of a cervical edema included in the cervical image region, and displays the boundaries thus detected in an emphasized function in the enlarged image.

16. The ultrasound diagnostic apparatus according to claim 1, further comprising:
a measuring unit that measures a cervical edema included in the cervical image region by detecting a longitudinal direction of the cervical edema, and measuring a distance between boundaries laid along a direction in perpendicular to the longitudinal direction thus detected, wherein
the display controlling unit controls to further display a measurement result of the measuring unit on the display device.

17. The ultrasound diagnostic apparatus according to claim 1, wherein the display controlling unit controls to further display a measurement result of a cervical edema that is manually measured on the enlarged image by a user onto the display device.

18. The ultrasound diagnostic apparatus according to claim 1, further comprising:
a determining unit that determines whether conditions of the fetus when the ultrasonic waves are transmitted and received satisfy conditions required to be satisfied for performing a cervical edema measurement, and
an alert output unit that outputs, when the determining units determines that the conditions are not satisfied, outputs an alert to a user.

19. A control method for an ultrasound diagnostic apparatus, the control method comprising:
extracting a cervical image region that is a region including a cervical region from an ultrasonic image of a fetus obtained by transmission and reception of ultrasonic waves;
detecting a dorsal body surface region that is a region related to a dorsal body surface of the fetus from the ultrasonic image; and
controlling to arrange an enlarged image including an enlarged image of the cervical image region in a region other than the dorsal body surface region included in the ultrasonic image, and to display the enlarged image onto a display device.

20. An image processing apparatus comprising:
an extracting unit configured to extract a cervical image region that is a region including a cervical region from an ultrasonic image of a fetus obtained by transmission and reception of ultrasonic waves;
a detecting unit configured to detect a dorsal body surface region that is a region related to a dorsal body surface of the fetus from the ultrasonic image; and
a display controlling unit configured to control to arrange an enlarged image including an enlarged image of the cervical image region in a region other than the dorsal body surface region included in the ultrasonic image, and to display the enlarged image onto a display device.
